(19) United States
(12) Patent Application Publication
(10) Pub. No.: US 2011/0065155 A1
(43) Pub. Date: Mar. 17, 2011

(54) Productions of Organic Acid Salts from Digested Biomass and Their Uses and Self Cleaning Emitter Therefor

(76) Inventor: Daniel D. De Lima, Westover, MD (US)

(21) Appl. No.: 12/843,847

(22) Filed: Jul. 26, 2010

Related U.S. Application Data
(60) Provisional application No. 61/264,304, filed on Nov. 25, 2009.
(60) Provisional application No. 61/228,640, filed on Jul. 27, 2009.

(51) Int. Cl. C12P 7/40

(52) U.S. Cl. 435/136

(57) ABSTRACT

A method and means of producing organic acids (such as acetic acid) and calcium-, magnesium-, iron-organic acid salts (mostly acetates) from readily available, digested biomass (or biomass rich trash or waste) and limestone, or magnesium rich stone, or iron oxides is described. The invention also relates to the use of inexpensive, common chemicals in a process for treating water and for the separation of microbes from fresh water or saltwater. The treatments include denitrification, flocculation-separation of microbes and suspended particles, and removing dissolved phosphates (i.e., those rendered insoluble by the process) from water. Other benefits of induced bio-flocculation include separating microbes for use as animal feed or separating fatty algae harvested in the production of bio-diesel. A two way emitter for use with the process is also described.

### CROSS REFERENCE TO RELATED APPLICATIONS

This application claims the benefit of U.S. Provisional Application 61/264,304, filed Nov. 25, 2009, entitled Productions of Organic Acid Salts from Digested Biomass and Their Uses, which is incorporated herein by reference. This application also claims the benefit of U.S. Provisional Application 61/228,640, filed Jul. 25, 2009, entitled Self Cleaning Filtering Emitter and New Prospects in Microbe Cultivation and Management, which is incorporated herein by reference.

### BACKGROUND OF THE INVENTION

### 1. Field of the Invention

the present application relates to human activity induced: (1) bio-flocculation and (2) phosphate stripping.

A method and means of producing organic acids (such as acetic acid) and calcium-, magnesium-, iron-organic acid salts (mostly acetates) from readily available, digested biomass (or biomass rich trash or waste) and limestone, or magnesium rich stone, or iron oxides is described. The invention also relates to the use of inexpensive, common chemicals in a process for treating water and for the separation of microbes from fresh water or saltwater. The treatments include denitrification, flocculation-separation of microbes and suspended particles, and removing dissolved phosphates (i.e., those rendered insoluble by the process) from water. Other benefits of induced bio-flocculation include separating microbes for use as animal feed or separating fatty algae harvested in the production of bio-diesel.

The denitrification may be by means known to those of ordinary skill in the art, such as adding organic carbon. However, the flocculation process is enabled by the microbes metabolizing the cationic part of an added solution, typically calcium and or magnesium acetate solution. The microbes metabolize the acetate, producing CO2 (carbonate and bicarbonate), then they or other microbes, metabolize the CO2 (by, for example, photosynthesis) to produce oxygen. The result is a solution rich with ions (calcium) without an equally strong cation (acetate or bicarbonate). The resulting Ca(OH)2, drastically increases the pH of the pond or tank holding the microbes. The increased pH renders the calcium and other polyvalent ions insoluble.

The dis-solution of these ions onto the surfaces on the suspended particles neutralizes separation-charges, creating defined boundaries to the particles, and will add to the weight of the suspended mass. These changes cause flocculation.

The increase in pH in the presence of phosphates with the presence of calcium, produce calcium phosphate in solid which co-precipitates with the microbes and other suspended micro-particulate matter.

Thus the deliberate addition of calcium acetate to microbes, particularly those that metabolize acetate (of which there are many), and the subsequent removal of the CO2 produced from the metabolizing of the acetate, results in flocculation and in co-precipitation.

### Background

Biomass is a cheap renewable source of carbon. Biomass intensive waste (anthropogenic refuges containing biomass) is often of negative value. Traditional flocculants such as ferric chloride (FeCl3) and aluminum sulfate Al2(SO4)3 by contrast are very costly. Calcium hydroxide is often used in tertiary water treatment's phosphate stripping. Ca(OH)2 is more costly than the limestone from which it was derived. Methanol, a carbon source used in denitrification in tertiary sewage treatment is expensive and, if produced from natural gas, adds to the CO2 in the atmosphere when used.

This invention relates to producing in situ inexpensive, weak acetic acid and mare, particularly cheap calcium acetate from biomass (and or biomass containing house hold garbage). The materials are often produced and collected in great quantities in towns (fall leaves, grass clippings, wet paper waste, etc). A bulk (about 70%) of these materials is returned to atmospheric CO2 when composted, and the approximate 30% residue is used as compost.

Biomas (i.e., bagasse) from sugar cane is produced in large quantities by sugar mills. Most is used to fuel the evaporation in the production of sugar. Some of the material is separated into fiber, and the fiber used to produce paper. The non-fiber (pith) is of low value since it has a low combustion-fuel value.

Thus consider the following:
(1) Wastewater treatment plants are usually close to towns, so transporting garden waste (leaves and grass clippings) close to these facilities will involve little cost change.
(2) Sugar cane and crops such as corn produce biomass crop residues that are not considered as sewage or waste, a legal use definition in that they contents are considered “generally regarded as safe (“GRAS”). This waste is found in farm areas that are often next to towns and cities. These cities and towns consume meat (chicken, pork, fish). Thus meat producing facilities (chicken houses and meat processing facilities) are also often in the farm areas close to towns and cities so as to enable staff to commute and to reduced the cost of product shipment to the nearby cities.
(3) Animal litter and pig fecal matter may be bio-digested to produce bio-gas and nutrient water. Biogas may be used as process fuel.
(4) Algae may be grown in nutrient water. Acetic acid may be used to pulse carbon-feed algae, and calcium acetate may be used to separate the algae from the water of cultivation. Thus, the production of acetic acid from crop residues and calcium acetate from crop residues and lime stone dust will enable the production of animal feeds (with calcium). These feeds will produce animal growth and closed cycle fecal matter. The animal growth may be used as meat.
(5) Diesel fuel used by trucks is mostly purchased where cheapest between routes that usually involve delivery to facilities close to cities. Thus, a facility that produces bio-diesel that is close to a city's waste water facility, will be close to a city and therefore close to where trucks buy their fuel, thus saving distribution costs.
(6) Acetate pulse feeding can increase the fat content of cultivated algae. Calcium acetate pulse feeding and auto-flocculation will enable cheap separation from water. Thus the proposed method uses waste leaves and house hold trash to produce acetic acid as carbon source, and uses waste leaves and household trash both digested with powdered limestone to produce calcium acetate, which will improve the yield of bio-fuel from algae and enable its cheaper separation.
(7) Fresh water may be ‘potable’ or ‘industrial’. Often ‘potable’ water is used for industry. The geography of the world's water supply is changing. Many human occupied areas of traditional rainfall (i.e., the US Southwest) will receive less rain fall in the predicted weather change patterns caused by global warming, whether the source of the change is caused by humans or not. Many mountain-valley glaciers will shrivel or melt completely so that their summer melt rate will greatly reduce are will disappear completely. This will greatly reduce stream flow in some areas. This stream flow volume is essential to agriculture in many parts of the world (e.g., India, China). Thus, the ability to cheaply recycle wastewater to industrial water, irrigation water or for aquifer recharge will be of significant value.
(8) In water recycling, this invention will enable denitrification by using acetic acid as a less costly and lower carbon footprint carbon source than methanol. It will enable the use of calcium that has been rendered insoluble by changed pH as a less costly flocculating agent, rather than using aluminum sulfate or ferric chloride. The process will enable the use of calcium acetate as an induced phosphate stripper in a tertiary wastewater treatment context where algae produces the oxygen to remove residual BOD, where microbes (including many algae) metabolize the acetate to produce growth and CO2, where the algae then removes the CO2 (bicarbonate), causing the pH to rise and rendering calcium phosphate very insoluble. The rendered insoluble salts change the balance of electric charge on microbes and other suspended solids surfaces causing flocculation and precipitation of microbes, suspended particulate solids and calcium phosphate. The result, after BOD removal, denitrification, phosphate stripping, and flocculation induced precipitation, is water that has been stripped of nearly all contamination. This water will be suitable for reuse (after filtration and ozonification for safety) and will have been very inexpensively rendered into this re-usable form.

This invention, among other benefits, will enable the production of meat (“feed” for animals) from crop residues, the production of bio fuel, and enable the recycling of water all with a minimal new-carbon footprint and all within a local community.

A very low cost source of acetic acid, and organic acid salts from biomass has and will have many other uses, especially in the future. As the world becomes sensitized to the production cost plus the environmental impact cost of adding fossil carbon to the atmosphere as CO2, enlightened governments will tax fossil fuels more and more. The increasing cost and limited supply of traditional bio-fuels (sugars, methanol, etc.) will encourage the use of new cheaper alternates such as organic acids and organic acid salts.

### SUMMARY OF THE INVENTION

Accordingly, it is a principal object of a preferred embodiment of the invention to provide one or more tanks to recycle common waste and fertilizer to produce one of biogas, feed, agchar and cleaned water.

It is another object of the invention to provide a biomass system that uses limestone and other lost cost materials in tanks that break down cellulose materials without requiring the high cost elements of earlier systems

It is a further object of the invention to provide a biomass system which is provided at a locale where the elements of the process are found to produce fuels and feeds at a site where they can be utilized, especially in warmer climates where energy costs are high.

Still another object of the invention is to provide a self sufficient and sustainable system that lowers carbon output in the production of fuels and feeds by recycling cellulose materials.

It is an object of the invention to provide improved elements and arrangements thereof in an apparatus for the purposes described which is inexpensive, dependable and fully effective in accomplishing its intended purposes.

These and other objects of the present invention will be readily apparent upon review of the following detailed description of the invention and the accompanying drawings. These objects of the present invention are not exhaustive and are not to be construed as limiting the scope of the claimed invention. Further, it must be understood that no one embodiment of the present invention need include all of the aforementioned objects or elements of the present invention. Rather, a given embodiment may include one or none of the aforementioned objects. Accordingly, these objects are not to be used to limit the scope of the claims of the present invention.

### BRIEF DESCRIPTION OF THE DRAWINGS

FIG. 1 is a view of an emitter according to at least one aspect of the invention in the normal position.

FIG. 2 is a view of the emitter in an open position.

FIG. 3 is a view of the emitter in the filter position.

FIG. 4 is a diagrammatic, side plan view of a first pond according to at least one aspect of the invention.

FIG. 5 is a diagrammatic, top view of a first pond according to at least one aspect of the invention.

FIG. 6 is a diagrammatic, side plan view of a second, shallow pond according to at least one aspect of the invention.

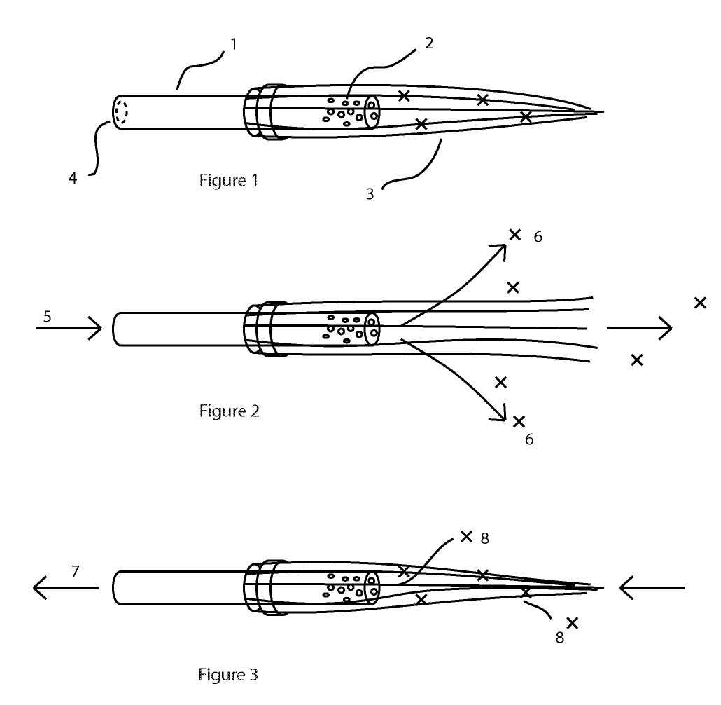

Similar reference characters denote corresponding features consistently throughout the attached drawings.

### DETAILED DESCRIPTION OF THE PREFERRED EMBODIMENT(S)

The invention is to a symbiotic digestion of cellulose-exposed biomass and limestone or other minerals in the production of organic acid salts.

Natural mature biomass, such as from sugar cane or other sources, normally has its cellulose encased in a lining of lignin. In the described process, it is assumed that the lignin encasement has been breached by known methods and that the cellulose of the biomass has been directly exposed to the interaction of chemicals, enzymes and microbes. This type of exposure may be produced by chemical means such as exposure to hydrogen peroxide in a alkaline medium, steam explosion where the biomass is heated with steam to about 180 Centigrade and the hot, steamed product forced through an orifice, or by fungal attack such as that of White rot fungus, where the fungus produces ligninase that penetrates the lignin protective layer and thereby exposes the cellulose to subsequent attach by other microbes. White rot fungus attack is the main biological activity at the early stages of composting.

In this invention, typically the exposed cellulose and powdered limestone (mixed within the biomass fibers) are submerged in water containing acid forming bacteria in a bio-digester (“reactor”). The reactor may be one such as that shown in U.S. Pat. Nos. 5,080,786 and 5,158,593 including a self cleaning two-way emitter as further described herein, including a transparent or other cover over the system. A collection area such as conical bottom surface or other similar device may be used to catch aggregates and precipitates at the bottom where they can be removed as will be described further hereinunder.

These bacteria attack the cellulose and produce weak organic acids (mostly acetic). In this process, the rate of the acid formation is inhibited by low pH. Thus as acids form and accumulate, the pH is lowered self-regulating the rate of additional acid formation.

To maintain a steady rate of acid formation, according to at least one aspect of this invention, as the acid is formed, it reacts with added limestone to form calcium acetate and CO2. In so doing, the pH is regulated and prevented from rising, and the rate of digestion is enhanced to a higher rate than if there were no limestone added.

Further, according to one aspect of the invention, suspended from a cover of the reactor and lying within the water submerged biomass limestone mix, are agitators (“vibrators”), say motors with eccentric cams that press on a diaphragm, the diaphragm being exposed to the media. These agitators are operated intermittently, such as for a few minutes every few hours. Their expansion wave is preferably fast and the compression wave is preferably slow (or vice versa) so as to send compression waves different to the contraction waves through the biomass mix. These pulses of expansion and compression cause fluid pumping because of different flow versus flex response of the media. This sets up small currents in the matrix. This flow results in fluid exchange between the enzyme attack site within the biomass fibers, and the limestone location site that may be a few microns away. The pumping action of the vibrations causes fluid that is low in pH to be transported to the limestone, where it reacts with the calcium and magnesium carbonate, liberating CO2 and increasing the pH. Then as the process continues, the higher pH carbonates migrates in random fashion back to the microbe-cellulose interaction site. Because of the increased pH, inhibition is lowered relative to that which would have been there had the carbonates not been included. Thus the extent of digestion is increased and the acetate concentration higher than that relative to what would have been produced had the limestone dust not been included.

After a period of digestion where even the higher Ca++salts cause inhibition, the free liquid may be pumped out of the reactor. The CO2 that has collected over the liquid is also evacuated, and atmospheric pressure compresses the fibers and squeezes out liquids trapped and in and around the fibrous mass (vacuum compression extraction).

In the voiding of the reactor of liquid through the emitters, the fiber covered opening of the two way self cleaning emitter, press onto one another to filter fluids exiting the reactor of particulates that may lodge in the supply tubes of the digester to prevent them from being clogged.

The bio-digester is refilled with water. The pH of the biomass within the digester returns to around 7. The dissolved calcium having been mostly removed in the previous vacuum compression extraction is greatly diluted by the introduced water. The cellulose has been attacked and broken down into acetic acid, loosing linear fibrous-ness and becoming more paste-like. As the mass is partly dissolved, the mass looses volume. The immediate condition of the cellulose acid-forming bacteria is of greatly reduced inhibition, and the digestion process begins again.

The acid forms again, and it is transported to the limestone by diffusion or by vibration pumping. The salts accumulate, the process eventually becomes inhibited by the salts though at a higher concentration than that had limestone not been mixed with the biomass. The biomass looses volume. It also looses linear structure, and becomes more pasty.

The digester is inflated so that the cover lifts off the surface of the contained biomass, fresh cellulose-exposed biomass is introduced by a blower and a rotary distributer and covers the previously deposited biomass from the previous ‘feeding’. The air that is introduced, and any air that may be bubbled in at ‘feeding’ time at the bottom of conical containment, inhibits methane-formers from establishing as methane formers are strict anaerobes, and a trace of oxygen kills them. The easily available biomass is thus converted to valuable organic acid or organic acid salts.

A quantity of pasty biomass that has migrated towards the bottom of the conical containment may be removed by pumping or other evacuation techniques.

To continue the process the voided, pasty biomass may contain some undigested cellulose. However, it may not be economical to continue to digest this cellulose as it may still be somewhat protected by lignin and the digestion rate would be very slow. This pasty residue may be pumped to a very-low-cost-per-unit-volume biogas digester. The residual biomass is maintained in strict anaerobic conditions to allow methane formers to operate on the biomass. The methane formers are established, and the residual cellulose is first converted to weak acid and then the weak acid is converted to biogas. The biogas is used to fuel the process.

From the biogas digester, a peat like paste is removed such as on a yearly basis. This paste is nearly pure lignin. The lignin may be charred and used as Agchar, a soil improver and a carbon-credit generator.

### Other Uses of Weak Acetic Acid and Weak Calcium Acetate

The process may be firstly used to produce additional products as part of a recycling process such as for a Water recycle, fertilizer recycle, and bio-fuel production from limestone and digested biomass. Secondly, the process may be used in the intense production of meat from sunlight, fertilizer, and the in situ production of cheap organic acid salts (calcium acetate) from crop residues, which that are key to the processor's economic efficiency to form feeds. Thirdly, in a similar cycle, the process may be used for the production of shrimp feed supplement and for the cleaning of shrimp aquaculture discharge water.

(1) Water recycle, fertilizer recycle, bio-fuel production.
a. The solids are separated from sewage. The solids are then sent to a biogas digester. The nutrient original liquid and the digester are used for algae cultivation. Garden trash may be burned to generate energy. The CO2 scrubbed and the scrubbing water is used as carbon in algae production. When the algae has used as many nutrients as possible, calcium acetate (organic acid from biomass) is added in the morning of a sunny day to initiate the next step in the process.
b. The algae metabolize the acetate, but not the calcium. The algae removes all the CO2 from the water. The pH rises and the calcium becomes insoluble. The insoluble calcium flocculates the algae (by coating the algae or other effects) and strips any residual phosphates.
c. The water may then be decanted, filtered and sterilized. It is then able to be used as commercial water or to recharge aquifers, etc.
d. The solids of sediment sent to the biogas digester produce biogas. The solids residue from the digester are dried and used as fertilizer. The liquid is dried and used as fertilizer. The biogas is CO2 stripped to make methane and the CO2 used as carbon supplement in algae growth.
   (2) Meat Production from Biomass.
a. Animal fecal matter and litter is digested to produce biogas and nutrient liquid.
b. CO2 from the biogas's combustion is used to CO2 feed algae.
c. Weak organic acids from biomass digestion and CO2 from biomass digestion is used as carbon source in algae growth.
d. Nutrient liquid from fecal matter digester and artificial fertilizer (with carbon from air, and from (b) and (c) are used also to grow algae.
e. When the algae tanks reach production/shading limit, calcium acetate from biomass with limestone digestion is added in the morning.
f. The algae metabolizes the organic acid. The algae metabolizes all the CO2 and thus the pH rises.
g. As the pH rises, the calcium becomes insoluble and precipitates the algae.
h. The water above the sediment is decanted. Some water is used (much less than the decanted amount) to jet the algae to catch-basins at the lower points in the membrane lined, algae cultivation ponds.
i. The algae and jetting/transport water is sent to a conical separator. Iron acetate is added and the suspension separates again. The water is decanted, and the solids sent to a vacuum filter. The solids from the vacuum filter are mixed with other ingredients and binders to make animal feed.
j. The feed may be used in animal production.
k. The animals grow and produce fecal matter (or litter). The process continues in closed loop.
   (3) Same as (1) but the algae used as shrimp feed supplement (not to produce biogas). Cooking of the feed may be used to keep viruses from being recycled.

A variation of the system could also be used for analogous food production such as Salmon farms, especially for in-land Salmon farms. Some of the steps could also be used to advantage in an open sea Salmon farm, where floating pens may be used in open sea.

One skilled in the art would appreciate that not each and every step need be used in every embodiment or a combination of steps from various embodiments without departing from the scope of the invention. For example, an in-land or near land system could be used to generate feed for the Salmon that would offset some of the waste produced by the Salmon farm.

### Self Cleaning Filtering Emitter and New Prospects in Microbe Cultivation and Management

A self cleaning filtering emitter for use with the above process may be made of the following: (1) An emitter-tube with one end 4 connected to a fluid distribution network of pipes. (2) The other end containing one or more holes. The emitting/collecting end is surrounded by fibers 3 made of plastic or other materials, preferably having a natural tendency to return to the position shown in FIG. 3. These fibers are attached (preferably by band and glue) at one end of the fiber to an area on the emitter tube between 1 and 2. Fluid flowing within 1 in the direction shown by arrow 5 spreads the fibers 3 and disperses biomass particles 6 that may be lodged thereon or caught within. This release of previously trapped particles cleans the filter. This is one way in the emitter mode.

In the event of reversal of flow (following arrow 7) (the other way in the emitter mode; a collector), biomass particles 8 that may be released by the body of biomass into the flow to the emitter, and may flow with the liquid, are trapped on the fibers of the emitter. This is in its collector mode. These particles lodge on or within the fibers and are filtered from the flow. The particles do not enter 2 and are thus prevented from choking the orifices or subsequent distribution/collection tubes.

The self-cleaning two way emitter is preferably deployed in a biomass digester described above, but may be used in other systems, where emitters discharge liquids into, and remove liquid from digesting biomass. The emitters should be aligned, loose fiberous end pointing along a direction parallel to the biomass's flow so that the fibers are cleaned and lay correctly during normal flow. For example, in the device shown in U.S. Pat. No. 5,158,591, the movement of biomass is sloping downward and thus the emitter would face downward.

Typically, digesting biomass within a digester have particles of various sizes and hardness. When a digester is having its liquid content removed from the biomass, many of these particles (or combination of particles) will lodge in and/or at the entrance to the ‘extracting orifice’. This will often lead to choking and will often render the ‘choked’ orifice useless. Also, prospects are that on the reversal of flow, the ‘choked’ orifice may not clear, as there may be one or more alternate ‘clean’ orifices on the same line where the resistance to flow is less, since they are “clean.” As clean orifices release the pressure necessary for clearing the choked orifice, the choked orifice remains choked. Since the choked orifice remains choked and the biomass in its proximity does not go through designed fluid exchange, the digestion's products are not extracted (or are extracted to a lesser extent). This undesirable choking of emitters will lead to prolonged fluid exchange and inefficiency.

The present self cleaning two-way emitter remedies the above problem and allows biomass digesters to operate more efficiently. The improved efficiency enables the following innovative way to cultivate and harvest microbes.

Cellulosic fibrous porous biomass when submerged (anoxic) in a digester, is often converted by many microbes to weak acetic acid (and other acids). If the cellulosic biomass is mixed with limestone, iron salts, etc, these minerals dissolve in the weak acid, and often enhance digestion by slowing pH reduction rate. Reduced pH often depresses acid formation.

The acetic acid and other organic acids are often metabolized by microbe. Some of these microbes may be algae. Also, in metabolism consumption, acetate salts have their acetate component metabolized by the microbes, leaving the ‘salt’ without an acetic component. The residual component after acetate metabolism, is often carbonate or hydroxide.

Thus, if acetic acid from digested biomass is used to feed algae and other microbes, these microbes will multiply and produce carbonate. If some (or all) of the microbes are algae, and the algae is exposed to adequate light, nutrients, etc, then the algae will metabolize the carbonate and grow more.

As the natural rate of algae growth in water is often limited by solution rate of atmospheric CO2, and the near total consumption of CO3=, and HCO3-, leads in hard Ca rich water to rising pH (from Ca(OH)2 formation), and as at high pH many salts are rendered insoluble, pulse feeding the algae/microbe mix with organic, slows flocculation. This enables to microbes to remain in suspension within the water column. The algae-microbe mix, being suspended in a light (illuminated) environment (algae adsorbing CO2 and liquid carbon without nutrients producing principally fat-growth (accumulation) and O2, or (algae adsorbing CO2 and nutrients, producing cell-growth and O2, and in symbiosis with the microbes (including many algae) adsorbing acetate, O2 and nutrients and producing cell growth, the feeding with organic acid stimulates growth and or fat formation while suppressing flocculation. This results in a rapid increase in microbial biomass.

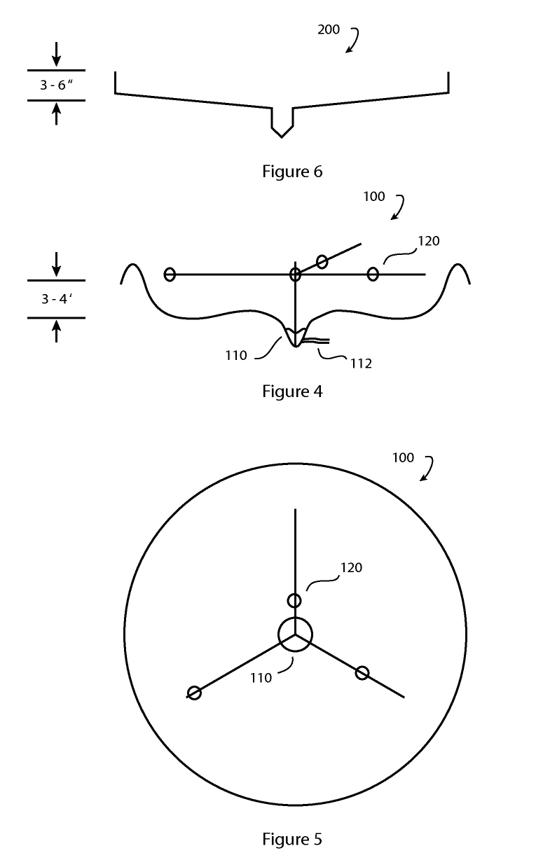

This increased biomass often slowly settles to the bottom of a cultivation pond such as that shown in the pond 100 (FIGS. 4-5). The settled microbial sediment can be easily washed with currents of water laterally directed to a central conical area 110. In this conical where there are no lateral or only very diminished lateral currents, the microbe biomass settles again. These settled microbes may be removed from the base of the conical system 110 as first stage concentration through a conduit 112 or the like. They may then be pumped to another pond 200 where they are fed calcium acetate. The microbes in light in a shallow pond then metabolize the acetate as described below.

The second, lined tank may be a shallow tank of for example 3 inches to 6 inches which allows sunlight to more easily affect the temperature of the water and allow for a less expensive system to further process the tank. The shallower tank results in warmer water, which results in faster microbial process and higher pH, which is good for scrubbing CO2 from the system and more settlement as compared to the 3 to 4 foot tank, which may have less heat and sunlight affecting the algae at depth. One skilled in the art would appreciate that liner vents, etc., could be used to prevent the bottom liner from “floating” up and working against the natural settling of sediments in the collection area 230. Because the temperature in the pond is higher and no acids are being added, flocculation tends to produce more stratified layers that can be collected below. If necessary, calcium magnesium acetate may be added to promote a high pH and further flocculation, since algae growth and microbial stability is not the focus of the second pond.

(A) In a system at pulse fed, enabled reduced pH, sufficient microbes and algae remain in the illuminated water column so as to enable symbiotic rapid microbial growth while sedimentation proceeds. This enables continuous microbial biomass production. However, during sunlight illumination, the upper strata of the water column becomes supersaturated with O2 and warm, inhibiting algae growth, whereas the lower strata become O2 starved. Mixing, typical of paddle wheel aerators 120 (that may be mounted on central pivoted floating rotating arms to rotate slowly around the pond), de-stratify the water column, which is “soupy” with flocculants, and produce temporary lateral currents over the area of the entire bottom. The aerators are run at a sufficient speed to mix the water to cull off new or excess growth and encourage the materials towards the catch funnel 110 without clearing out helpful algae and chemicals necessary to continue the process. Thus concentration by lateral transport of the sediment and desertification can be achieved by a single effort input of the mixer.

(B) During the night, many microbes continue to consume O2, as the algae at night do not produce O2, the dissolved oxygen in the water column becomes depleted. Aeration by paddle wheel aerators (that may be mounted on central pivoted floating rotating arms) may also be used to dissolve atmospheric O2 into the water column and to stave off anaerobic microbes as discussed above. These aerators produce temporary lateral currents over the area of the entire bottom. The lateral flow may be coincidentally directed so that the flow laterally washes the settled algae towards a conical depressions/collection point. Thus a single energy input achieves two objectives.

If upon sufficient growth, the algae (and others) are fed salts of acetate (example calcium acetate or iron acetate), the microbes metabolize the acetate. Then the algae metabolize the carbonate produced therefrom. The result is typically a hydroxide of the calcium or iron, and a rise in pH. Many salts are less soluble at high pH.

The minerals (typically calcium and iron) are rendered insoluble in hydroxide form by the rise in pH, and the mineral often coat the microbes and flocculate them. The relative high specific gravity of the minerals added to the microbes (near one specific gravity) enables the flocculated microbes to precipitate downward. The precipitate may be bushed to a location where it is harvested, or the fluid where in the microbes were suspended may be decanted, leaving a microbe and mineral co-precipitate as sludge on the bottom of the container.

This sludge may be washed to a drain inlet and removed. More microbes may be cultivated in the container (or added to the container) and so on.

Such flocculate microbes in an alkaline matrix of calcium may be putrefied, so the ammonia is liberated from the protein. The ammonia (in alkaline environment) may be stripped of by heating and by bubbling air through the putrefied biomass. The ammonia may be recovered, and the ammonia stripped putrefied biomass may be used to produce biofuels without ammonia inhibition.

The flocculated microbes may be processed to disrupt cells' membranes such that the cells' contents may be recovered and put to uses. These uses include to recovery of pigments and exotic substances.

(c) The removed sludge may be filtered and otherwise dewatered. One use is to mix the dewatered sludge with a binding carbohydrate (flour) and to heat (disrupt cells by cooking or enzymes) and palletize the mixture. Such pellets would contain microbes' (protein, lipids, etc), carbohydrates (flour), and the minerals (calcium and iron). Such pellets may be an economic form of animal feed supplement.

While this invention has been described as having a preferred design, it is understood that it is capable of further modifications, uses and/or adaptations of the invention following in general the principle of the invention and including such departures from the present disclosure as come within the known or customary practice in the art to which the invention pertains and as maybe applied to the central features hereinbefore set forth, and fall within the scope of the invention and the limits of the appended claims. It is therefore to be understood that the present invention is not limited to the sole embodiment described above, but encompasses any and all embodiments within the scope of the following claims.

I claim:

1. A method and means of producing organic acids comprising:
providing a pool of water;
adding organic acid salts from biomass to the water;
adding calcium-, magnesium and iron to the water;
adding limestone to limit acidity of the pool;
wherein the mixture denitrifies the water and flocculation-separation of microbes and suspended particles, and removes dissolved phosphates from the water.

2. The method of claim 1, wherein the flocculated suspended particles are used to produce animal feed.

3. The method of claim 1, wherein the flocculated suspended particles are used to produce bio-diesel.
